(19) United States
(12) Patent Application Publication
(10) Pub. No.: US 2016/0073947 A1
(43) Pub. Date: Mar. 17, 2016

(54) MANAGING COGNITIVE ASSISTANCE

(71) Applicant: Glen J. Anderson, Beaverton, OR (US)

(72) Inventor: Glen J. Anderson, Beaverton, OR (US)

(21) Appl. No.: 14/488,524

(22) Filed: Sep. 17, 2014

(51) Int. Cl. A61B 5/00; A61B 5/0476; A61B 5/04

(52) U.S. Cl. CPC A61B 5/4088; A61B 5/04008; A61B 5/0075; A61B 5/486; A61B 5/747; A61B 5/0002; A61B 5/7282; A61B 5/0476; A61B 5/6898

(57) ABSTRACT

Technologies for managing cognitive assistance provided to a user of a cognitive assistance system include a cognitive assistance system to determine a cognitive state of the user based on sensor data generated by one or more biosensors of the cognitive assistance system. The cognitive assistance system determines whether the user requires assistance based on the determined cognitive state of the user and identifies, in response to determining the user requires assistance, a trusted mobile computing device within a vicinity of the cognitive assistance system based on a trust relationship previously established between the cognitive assistance system and the trusted mobile computing device. The cognitive assistance system further communicates with the trusted mobile computing device to notify a remote user of the trusted mobile computing device that the user requires assistance.

### BACKGROUND

With an aging population worldwide, the number of individuals with some level of cognitive impairment continues to grow. For example, by the year 2020, an estimated seventy million people will have some level of dementia. Additionally, elderly persons oftentimes have other types of cognitive impairment at various stages. In general, an individual's cognitive decline is very gradual, so assistance with certain tasks the individual finds difficult can extend the years of independence and quality of life of the individual significantly.

A wide array of technologies (e.g., sophisticated brain computer interfaces) permit biophysical signals and characteristics to be sensed and interpreted by a computing device. For example, based on temporal and spatial patterns of biophysical signals obtained though electrical, optical, fluidic, and/or magnetic sensing devices, a computing device may measure and identify psychological states and/or mental representations of a person.

### BRIEF DESCRIPTION OF THE DRAWINGS

The concepts described herein are illustrated by way of example and not by way of limitation in the accompanying figures. For simplicity and clarity of illustration, elements illustrated in the figures are not necessarily drawn to scale. Where considered appropriate, reference labels have been repeated among the figures to indicate corresponding or analogous elements.

FIG. 1 is a simplified block diagram of at least one embodiment of a system for managing cognitive assistance provided to a user of a wearable computing device;

FIG. 2 is a simplified block diagram of at least one embodiment of an environment of a wearable computing device of the system of FIG. 1; and

FIGS. 3-4 is a simplified flow diagram of at least one embodiment of a method of managing cognitive assistance provided to a user of the wearable computing device of the system of FIG. 1.

### DETAILED DESCRIPTION OF THE DRAWINGS

While the concepts of the present disclosure are susceptible to various modifications and alternative forms, specific embodiments thereof have been shown by way of example in the drawings and will be described herein in detail. It should be understood, however, that there is no intent to limit the concepts of the present disclosure to the particular forms disclosed, but on the contrary, the intention is to cover all modifications, equivalents, and alternatives consistent with the present disclosure and the appended claims.

References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described. Additionally, it should be appreciated that items included in a list in the form of “at least one A, B, and C” can mean (A); (B); (C): (A and B); (B and C); (A and C); or (A, B, and C). Similarly, items listed in the form of “at least one of A, B, or C” can mean (A); (B); (C): (A and B); (B and C); (A and C); or (A, B, and C).

The disclosed embodiments may be implemented, in some cases, in hardware, firmware, software, or any combination thereof. The disclosed embodiments may also be implemented as instructions carried by or stored on one or more transitory or non-transitory machine-readable (e.g., computer-readable) storage medium, which may be read and executed by one or more processors. A machine-readable storage medium may be embodied as any storage device, mechanism, or other physical structure for storing or transmitting information in a form readable by a machine (e.g., a volatile or non-volatile memory, a media disc, or other media device).

In the drawings, some structural or method features may be shown in specific arrangements and/or orderings. However, it should be appreciated that such specific arrangements and/or orderings may not be required. Rather, in some embodiments, such features may be arranged in a different manner and/or order than shown in the illustrative figures. Additionally, the inclusion of a structural or method feature in a particular figure is not meant to imply that such feature is required in all embodiments and, in some embodiments, may not be included or may be combined with other features.

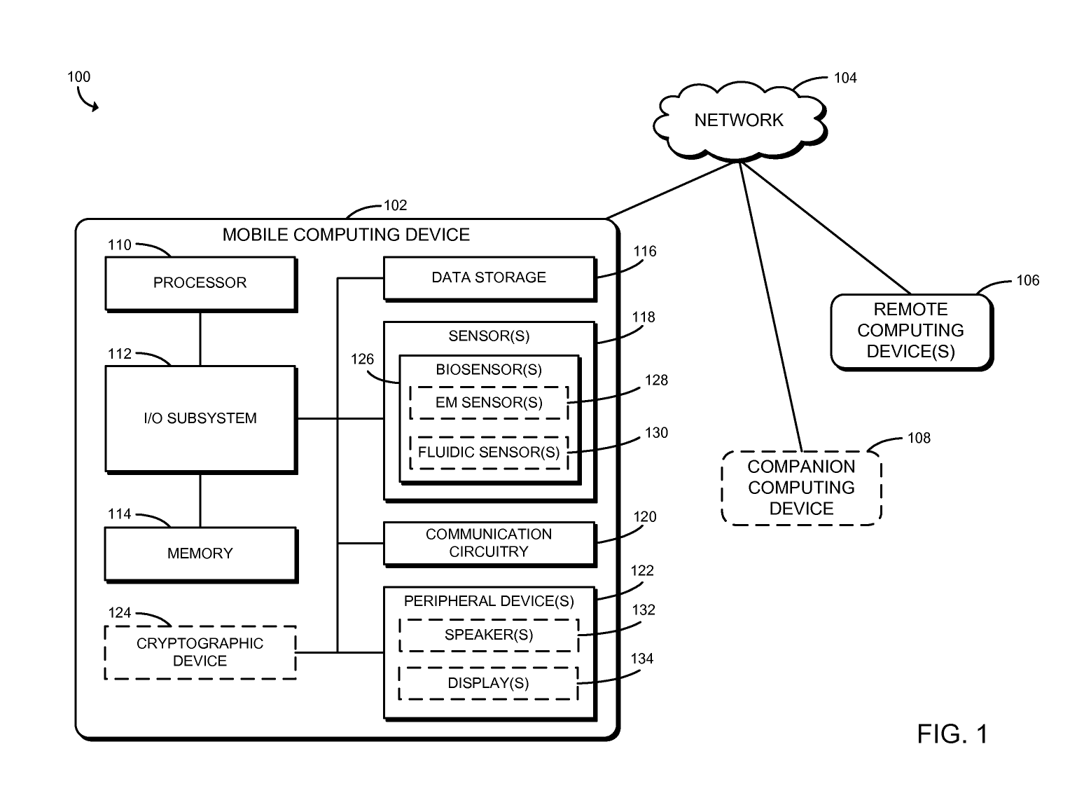

Referring now to FIG. 1, an illustrative system 100 for managing cognitive assistance provided to a user of a mobile computing device 102 includes the mobile computing device 102, a network 104, and one or more remote computing devices 106. Additionally, in some embodiments, the system 100 may include a companion computing device 108 as shown in FIG. 1. In use, as discussed in more detail below, the mobile computing device 102 determines a cognitive state of the user of the mobile computing device 102 based on data generated by one or more sensors 118 of the mobile computing device 102. The mobile computing device 102 further determines whether the user requires assistance based on the determined cognitive state of the user and, if so, the type of assistance required by the user (e.g., emergency assistance, assistance from the mobile computing device 102 itself, assistance from a nearby person such as a trusted person, etc.). If the user requires assistance from another person, the mobile computing device 102 communicates with one or more remote computing devices 106 (e.g., trusted computing devices) and/or remote users near the mobile computing device 102 (e.g., within a reference range of the mobile computing device 102). For example, as discussed below, the mobile computing device 102 may establish a trust relationship with a remote computing device 106 so that the mobile computing device 102 may securely communicate with the trusted remote computing device 106 when the user requires assistance.

The mobile computing device 102 may be embodied as any type of mobile computing device capable of being worn by a user and performing the various functions described herein. For example, the mobile computing device 102 may be embodied as a smartphone, personal digital assistant, tablet computer, laptop computer, notebook, netbook, ultrabook™, mobile Internet device, wearable computing device, and/or any other mobile computing/communication device. In the illustrative embodiment, the mobile computing device 102 is embodied as a wearable computing device, and the wearable computing device may further be embodied as, or otherwise include, a type of head-mounted display (e.g., computer eyewear), an earpiece, a bone-conducting speaker, and/or another wearable computing device capable of performing the functions described herein. In some embodiments, the mobile computing device 102 includes a companion computing device 108 with which the mobile computing device 102 is configured to communicate to perform the functions described herein. For example, in some embodiments, the mobile computing device 102 may be embodied as a wearable computing device configured to collect sensor data, which is transmitted to the companion computing device 108 for analysis.

As shown in FIG. 1, the illustrative mobile computing device 102 includes a processor 110, an input/output (“I/O”) subsystem 112, a memory 114, a data storage 116, one or more sensors 118, a communication circuitry 120, and one or more peripheral devices 122. Additionally, in some embodiments, the mobile computing device 102 may include a cryptographic device 124 to facilitate cryptographic functions, such as secure pairing and communications. Of course, the mobile computing device 102 may include other or additional components, such as those commonly found in a typical computing device (e.g., various input/output devices and/or other components), in other embodiments. Additionally, in some embodiments, one or more of the illustrative components may be incorporated in, or otherwise from a portion of, another component. For example, the memory 114, or portions thereof, may be incorporated in the processor 110 in some embodiments.

The processor 110 may be embodied as any type of processor capable of performing the functions described herein. For example, the processor 110 may be embodied as a single or multi-core processor(s), digital signal processor, microcontroller, or other processor or processing/controlling circuit. Similarly, the memory 114 may be embodied as any type of volatile or non-volatile memory or data storage capable of performing the functions described herein. In operation, the memory 114 may store various data and software used during operation of the mobile computing device 102 such as operating systems, applications, programs, libraries, and drivers. The memory 114 is communicatively coupled to the processor 110 via the I/O subsystem 112, which may be embodied as circuitry and/or components to facilitate input/output operations with the processor 110, the memory 114, and other components of the mobile computing device 102. For example, the I/O subsystem 112 may be embodied as, or otherwise include, memory controller hubs, input/output control hubs, firmware devices, communication links (i.e., point-to-point links, bus links, wires, cables, light guides, printed circuit board traces, etc.) and/or other components and subsystems to facilitate the input/output operations. In some embodiments, the I/O subsystem 112 may form a portion of a system-on-a-chip (SoC) and be incorporated, along with the processor 110, the memory 114, and other components of the mobile computing device 102, on a single integrated circuit chip.

The data storage 116 may be embodied as any type of device or devices configured for short-term or long-term storage of data such as, for example, memory devices and circuits, memory cards, hard disk drives, solid-state drives, or other data storage devices. The data storage 116 and/or the memory 114 may store various data during operation of the mobile computing device 102 such as, for example, cryptographic keys, identification data for trusted remote computing devices 106, threshold data, assistance classification data, and/or other data useful in the operation of the mobile computing device 102 as discussed below.

The sensors 118 generate sensor data regarding a user of the mobile computing device 102, the environment of the mobile computing device 102, the mobile computing device 102 itself, and/or other data useable by the mobile computing device 102 in determining, for example, a cognitive state of the user. As shown in the illustrative embodiment, the sensors 118 illustratively include one or more biosensors 126, which may be embodied as, for example, electromechanical sensors 128 and/or fluidic sensors 130. As discussed below, the biosensors 126 are configured to generate sensor data indicative of a cognitive state of the user based on one or more biophysical characteristics of the user of the mobile computing device 102.

In the illustrative embodiment of FIG. 1, the electromechanical sensors 128 may sense electromagnetic physical activity of the user. For example, in some embodiments, the electromechanical sensors 128 are configured to sense data to be used with electroencephalography (EEG) and/or magnetoencephalography (MEG). The fluidic sensors 130 may sense data (e.g., via optical sensing) to be used with functional near-infrared spectroscopy (fNIRS) for functional neuroimaging in some embodiments. In the illustrative embodiment, the biosensors 126 may be embodied as, or otherwise include, any sensors configured to sense data that may be analyzed or processed to identify, for example, psychological states or mental representations of the user, cognitive workload of the user, a level of attention or distraction of the user, the user's mood, sociological dynamics associated with the user, user memories, and/or other biophysical characteristics associated with the cognitive state of the user (e.g., heart rate, brain activity, etc.).

It should be appreciated that, in some embodiments, the sensors 118 may be embodied as, or otherwise include, other sensors to sense data used for face/object detection and recognition, determining a context of the user (e.g., determining a user activity such as whether the user is walking, running, or talking with someone), evaluating the physical environment of the mobile computing device 102, and/or identifying gestures, posture, voice, eye-tracking, facial expressions, and/or other inputs from the user and/or remote users. In various embodiments, the sensors 118 may be embodied as, or otherwise include, for example, proximity sensors, optical sensors, light sensors, audio sensors, temperature sensors, motion sensors, piezoelectric sensors, cameras, and/or other types of sensors. Of course, the mobile computing device 102 may also include components and/or devices configured to facilitate the use of the sensor(s) 118. It should be appreciated that the sensors 118 may be located on the mobile computing device 102 or elsewhere on the user (e.g., embedded in the user's clothes) and communicatively coupled to the main portion of the mobile computing device 102.

The communication circuitry 120 of the mobile computing device 102 may be embodied as any communication circuitry, device, or collection thereof, capable of enabling communications between the mobile computing device 102 and other remote devices (e.g., the remote computing devices 106). The communication circuitry 120 may be configured to use any one or more communication technology (e.g., wireless or wired communications) and associated protocols (e.g., Ethernet, Bluetooth®, Wi-Fi®, WiMAX, etc.) to effect such communication.

In some embodiments, the mobile computing device 102 may also include one or more peripheral devices 122. For example, as shown in FIG. 1, the peripheral devices 122 may include one or more speakers 132 and/or one or more displays 134. Each of the speakers 132 may be embodied as any device or components configured to generate a sound audible to the user of the mobile computing device 102 and/or persons in the vicinity of the mobile computing device 102. For example, in some embodiments, a speaker 132 may be embodied as a bone-conducting speaker. Each of the displays 134 may be embodied as any one or more display screens on which information may be displayed to a user of the mobile computing device 102. The display 134 may be embodied as, or otherwise use, any suitable display technology for doing so. For example, in some embodiments, the display 134 is embodied as a projection camera and associated projection surface mounted on a pair of eyeglasses (e.g., a transparent projection surface). In other embodiments, the display 134 may be embodied as some other combination of a projector and corresponding projection surface. For example, in some embodiments, images may be projected directly into the user's eye. Further, in some embodiments, the display 134 may be embodied as, or otherwise use, any suitable display technology including, for example, a liquid crystal display (LCD), a light emitting diode (LED) display, a cathode ray tube (CRT) display, a plasma display, and/or other display technology. The peripheral devices 122 may include any number of additional or other peripheral or interface devices (e.g., tactile devices), and the particular devices included in the peripheral devices 122 may depend on, for example, the type and/or intended use of the mobile computing device 102.

The cryptographic device 124 may be embodied as any hardware component(s) or circuitry capable of performing cryptographic functions and/or establishing a trusted execution environment. For example, in some embodiments, the cryptographic device 124 may be embodied as a security co-processor, such as a trusted platform module (TPM), or an out-of-band processor. Additionally, in some embodiments, the cryptographic device 124 may establish an out-of-band communication link with remote devices.

The network 104 may be embodied as any type of communication network capable of facilitating communication between the mobile computing device 102 and remote devices (e.g., the remote computing devices 106). In the illustrative embodiment, the network 104 is embodied as a personal area network (PAN) or an ad hoc network. However, in other embodiments, the network 104 may be embodied as, or otherwise include local or wide area networks such as, for example, one or more cellular networks, telephone networks publicly available global networks (e.g., the Internet), or any combination thereof. As such, the network 104 may include one or more networks, routers, switches, computers, and/or other intervening devices.

Each of the remote computing devices 106 and/or the companion computing device 108 may be embodied as any type of computing device capable of performing the functions described herein. For example, in some embodiments, the remote computing devices 106 and/or the companion computing device 108 may be similar to the mobile computing device 102 as described above. In the illustrative embodiment, each of the remote computing devices 106 is embodied as a mobile computing device of a remote user, such as a smart phone or tablet computer. Additionally, in embodiments including the companion computing device 108, the companion computing device 108 may be embodied as, for example, another mobile computing device of the user. For example, in some embodiments, the mobile computing device 102 may be embodied as a wearable computing device and the companion computing device 108 may be embodied as a smartphone. In other embodiments, each of the remote computing devices 106 may be embodied as a desktop computer, server, laptop computer, notebook, netbook, ultrabook™, personal digital assistant, mobile Internet device, wearable computing device, Hybrid device, and/or any other computing/communication device. Similarly, the companion computing device 108 may be embodied as any suitable mobile computing device (e.g., a mobile computing device on the user's person). Further, the remote computing devices 106 and/or the companion computing device 108 may include components similar to those of the mobile computing device 102 discussed above. The description of those components of the mobile computing device 102 is equally applicable to the description of components of the remote computing devices 106 and the companion computing device 108 and is not repeated herein for clarity of the description. Further, it should be appreciated that any of the remote computing devices 106 and/or the companion computing device 108 may include other components, sub-components, and devices commonly found in a computing device, which are not discussed above in reference to the mobile computing device 102 and not discussed herein for clarity of the description.

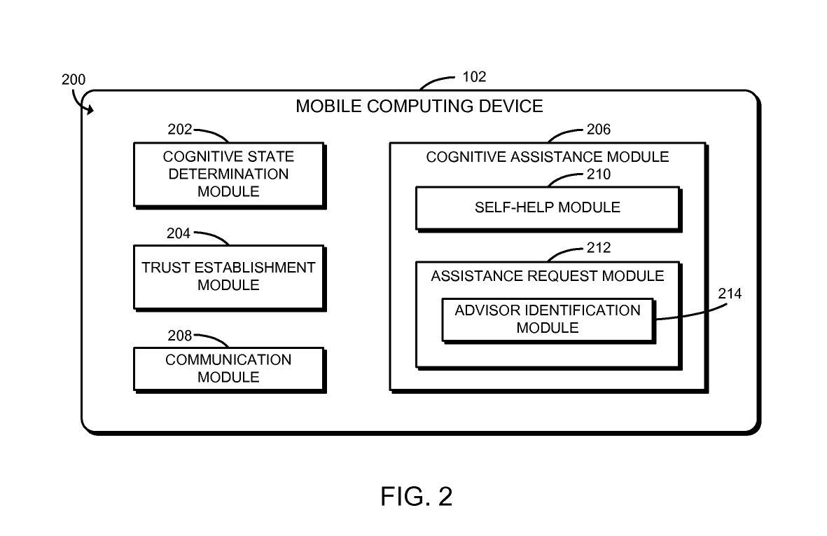

Referring now to FIG. 2, in use, the mobile computing device 102 establishes an environment 200 for managing cognitive assistance provided to a user of the mobile computing device 102. As discussed below, the mobile computing device 102 determines a cognitive state of the user based on data generated by the sensors 118 and determines the type of assistance required, if any, by the user of the mobile computing device 102 based on the user's cognitive state. If the user requires assistance from another person, the mobile computing device 102 communicates with one or more remote computing devices 106 and/or remote users within a reference range of the mobile computing device 102 to notify the remote user that the user of the mobile computing device 102 requires assistance.

The illustrative environment 200 includes a cognitive state determination module 202, a trust establishment module 204, a cognitive assistance module 206, and a communication module 208. Additionally, as shown, the cognitive assistance module 206 includes a self-help module 210 and an assistance request module 212, and the assistance request module 212 includes an advisor identification module 214. Each of the modules of the environment 200 may be embodied as hardware, software, firmware, or a combination thereof. For example, each of the modules, logic, and other components of the environment 200 may form a portion of, or otherwise be established by, the processor 110 of the mobile computing device 102.

The cognitive state determination module 202 determines a cognitive state of the user based on the sensor data generated by the sensors 118 (e.g., by the biosensors 126) of the mobile computing device 102. That is, the cognitive state determination module 202 analyzes the generated sensor data to determine various biophysical characteristics of the user (e.g., heart rate, brain activity, blood pressure, temperature, etc.), which is utilized to determine the cognitive state of the user. For example, as indicated above, the cognitive state determination module 202 may determine psychological or mental states of the user, a cognitive workload of the user, a level of attention or distraction of the user, the user's mood, sociological dynamics associated with the user, user memories, and/or biophysical characteristics relevant in determining the cognitive state of the user. It should be appreciated that, in some embodiments, the cognitive state determination module 202 may communicate with a remote computing device 106 (via the communication module 208) to transmit the generated sensor data and/or a processed version thereof for remote analysis in determining the user's cognitive state.

The trust establishment module 204 establishes a trust relationship with one or more of the remote computing devices 106. For example, the user of the mobile computing device 102 may trust certain persons (e.g., family member, caregiver, etc.) with assisting her in times of cognitive difficulty more than others (e.g., common passersby). Accordingly, the trust establishment module 204 may establish a trust relationship with the computing device 106 of the trusted person such that the mobile computing device 102 may subsequently securely communicate with the trusted user's computing device 106. In some embodiments, the trusted person (via their computing device 106) may utilize a “power of attorney” interface that allows the trusted person to securely communicate with the user of the mobile computing device 102, update data (e.g., schedules) on the mobile computing device 102, and/or otherwise control one or more functions of the mobile computing device 102.

It should be appreciated that the trust establishment module 204 may establish the trust relationship using any suitable techniques, algorithms, and/or mechanisms. For example, in some embodiments, the mobile computing device 102 and the trusted computing device 106 exchange cryptographic keys or otherwise establish a secure pairing between the devices 102, 106. In doing so, the computing devices 102, 106 may utilize any suitable cryptographic algorithms depending on the particular embodiment. Further, in some embodiments, the mobile computing device 102 stores identification data of the trusted computing device 106, which the mobile computing device 102 may subsequently use to identify the trusted computing device 106 (e.g., to distinguish the trusted remote computing devices 106 from untrusted remote computing devices 106). For example, the mobile computing device 102 may store an International Mobile Station Equipment Identity (IMEI) and/or cryptographic keys of the trusted remote computing device 106. It should be appreciated that, in some embodiments, the trust establishment module 204 may be embodied as, or established on, the cryptographic device 124. As described herein, in other embodiments, the mobile computing device 102 or the companion computing device 108 may determine the identity of a person in the vicinity of the mobile computing device 102 using one or more of the sensors 118. For example, the computing device 102, 108 may utilize audiovisual sensors to analyze the environment of the computing device 102, 108 and to identify persons in the vicinity of the computing device 102, 108.

The cognitive assistance module 206 determines whether the user of the mobile computing device 102 requires assistance based on the cognitive state of the user at any given point in time. As described herein, if the user requires assistance, the cognitive assistance module 206 determines the type of assistance required or otherwise determines an appropriate manner by which to address any cognitive difficulties the user may be having. In some embodiments, the cognitive assistance module 206 compares the sensed biophysical characteristics of the user and/or derived or processed values therefrom to one or more thresholds associated with types of assistance required. In a relatively simple example, the cognitive assistance module 206 may consider the user's heart rate and various thresholds. If the user's heart rate increases mildly (e.g., up to a specific threshold), the cognitive assistance module 206 may determine that no assistance is needed from other persons (e.g., the mobile computing device 102 can handle the user's problem). If the user's heart rate has increased significantly (e.g., beyond the threshold) and/or other biophysical characteristics indicate significant stress, the cognitive assistance module 206 may determine assistance is needed from another person (e.g., a trusted person). Further, if the user's heart rate drops significantly (e.g., below a threshold), the cognitive assistance module 206 may determine there is an emergency situation and alert everyone in the vicinity of the mobile computing device 102. As discussed above, the cognitive assistance module 206 includes the self-help module 210 and the assistance request module 212.

The self-help module 210 is configured to provide cognitive assistance to the user (e.g., if assistance is possible without intervention from another person). Depending on the particular embodiment, the self-help module 210 may, for example, display a message on the display(s) 134 of the mobile computing device 102 and/or render an audible message on the speaker(s) 132 of the mobile computing device 102 directed to assisting the user based on the cognitive state of the user. For example, in an embodiment, the cognitive state determination module 202 may determine that the user is confused and may also identify the user's niece (e.g., via facial recognition) in front of the user. As such, the mobile computing device 102 may determine the user may have forgotten his niece's name, so the self-help module 210 renders (e.g., via audio and/or image(s)) the niece's name and/or other information regarding the niece for the user. In some embodiments, the displayed image(s) and/or audio may only be sensed by the user (e.g., by virtue of a bone-conducting speaker, retinal projection, ear piece, eyeglasses with integrated displays, etc.). In other embodiments, the self-help module 210 may communicate with the user in other suitable ways.

The assistance request module 212 identifies remote computing devices 106 and/or persons near the mobile computing device 102. It should be appreciated that, depending on the particular embodiment, the distance (e.g., a reference distance) within which devices 106 are considered to be nearby or within the vicinity of the mobile computing device 102 may vary. For example, such a reference distance may be based on the communication range of the communication circuitry 120 and/or the particular communication protocol (e.g., Bluetooth™) used by the communication circuitry 120. The assistance request module 212 is further configured to request assistance from one or more of the remote computing devices 106 and/or persons. As described below, depending on the circumstances (e.g., emergency or non-emergency situation), the assistance request module 212 may request assistance from a trusted person/device or untrusted person/device of the user of the mobile computing device 102. Additionally, the assistance request module 212 may request assistance by virtue of communicating with the remote computing devices 106 in the vicinity of the mobile computing device 102 and/or audibly through the speakers 132 (and/or other output components) of the mobile computing device 102 depending on the particular circumstances.

The advisor identification module 214 determines the person(s) and/or remote computing device(s) 106 from which to request assistance. In doing so, the advisor identification module 214 may determine whether any trusted computing devices 106 are near the mobile computing device 102 using, for example, the stored identification data and/or cryptographic keys associated with the computing devices 106 with which the mobile computing device 102 had previously established a trust relationship. For example, the advisor identification module 214 may request or otherwise identify (i.e., determine the identification data for) the computing devices 106 in the vicinity of the mobile computing device 102 (e.g., within a predefined communication range) and compare that data to the stored identification data. In some embodiments, the advisor identification module 214 may, additionally or alternatively, scan the surroundings of the mobile computing device 102 to identify any persons identified by the mobile computing device 102 as trusted persons (e.g., via facial recognition).

The communication module 208 handles the communication between the mobile computing device 102 and remote computing devices (e.g., the remote computing devices 106) through the network 104. Accordingly, the communication module 208 is configured to establish a secure communication channel between the mobile computing device 102 and any trusted computing devices 106 and to exchange instructions and/or other information between the mobile computing device 102 and the computing devices 106. Further, in some embodiments, the communication module 208 is configured to transmit generated sensor data to a remote computing device 106 for a determination of the cognitive state of the user. In some embodiments, the companion device 108 and/or a remote computing device 106 may include one or more modules of the environment 200 (e.g., the cognitive state determination module 202, the trust establishment module 204, and/or the cognitive assistance module 206) for offloaded execution.

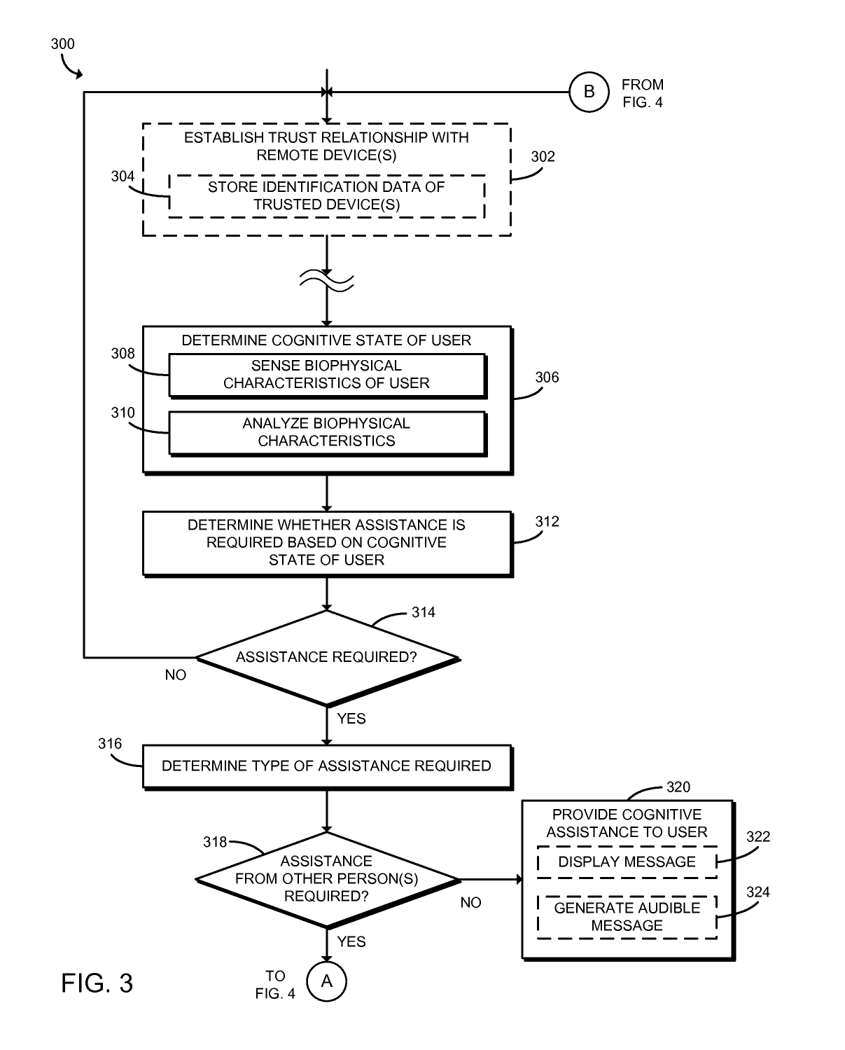

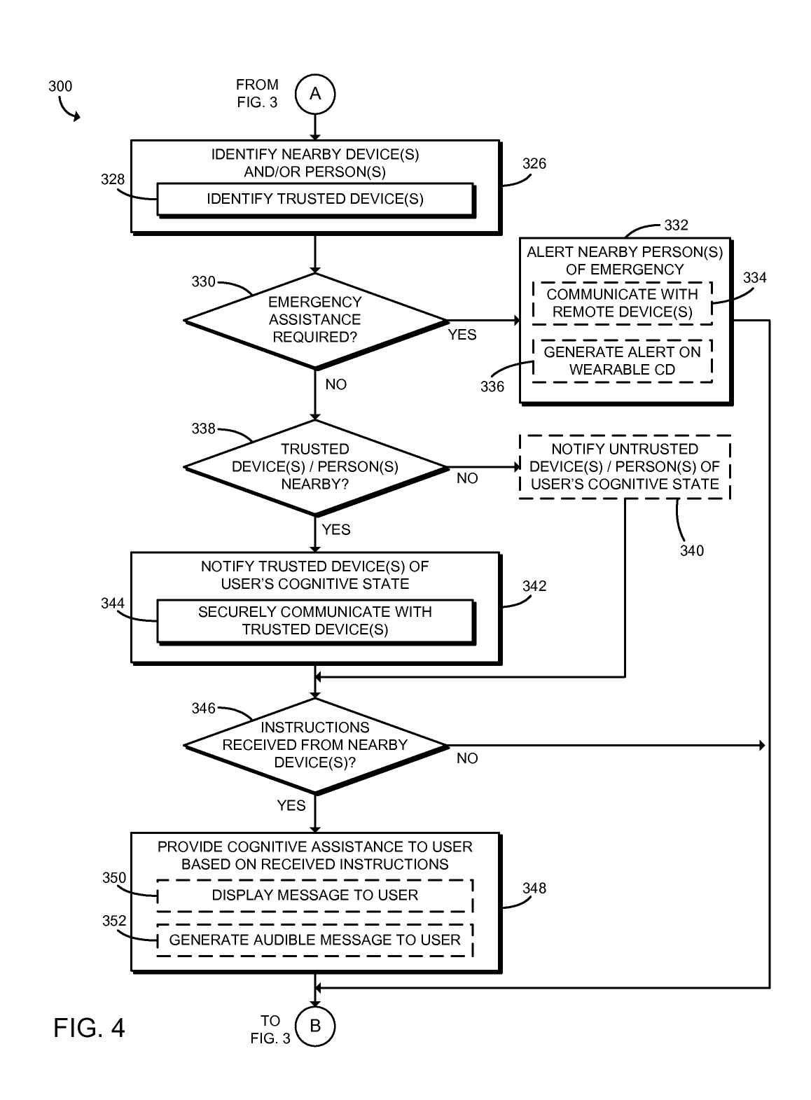

Referring now to FIGS. 3-4, in use, the mobile computing device 102 may execute a method 300 of managing cognitive assistance provided to a user of the mobile computing device 102. The illustrative method 300 begins with block 302 of FIG. 3 in which the mobile computing device 102 may establish a trust relationship with one or more remote computing devices 106. As discussed above, the mobile computing device 102 may utilize any suitable techniques for doing so. For example, the mobile computing device 102 may generate and/or exchange cryptographic keys with a particular remote computing device 106 to establish a secure pairing between the two devices 102, 106. In some embodiments, the mobile computing device 102 and the remote computing device 106 must be in close proximity, utilize a shared password, and/or perform some other action to ensure the secure pairing is legitimate. Further, in block 304, the mobile computing device 102 may receive/access and store identification data (e.g., IMEI) of the trusted computing devices 106. As discussed below, the identification data may be used by the mobile computing device 102 to identify trusted computing devices 106 when the user of the mobile computing device 102 requires cognitive assistance. It should be appreciated that, in some embodiments, the mobile computing device 102 may establish and/or cancel trusted relationships at any point in time.

In block 306, the mobile computing device 102 determines the cognitive state of the user. As discussed above, in doing so, the mobile computing device 102 may sense various biophysical characteristics of the user in block 308 and analyze those biophysical characteristics in block 310. It should be appreciated that the particular biophysical characteristics sensed may vary depending on the particular embodiment. Further, the mobile computing device 102 may utilize any suitable techniques, algorithms, and/or mechanisms for analyzing the sensed biophysical characteristics and for determining the cognitive state of the user. For example, in some embodiments, the mobile computing device 102 may utilize EEG, MEG, fNIRS, and/or other techniques to determine, for example, psychological or mental states of the user, a cognitive workload of the user, a level of attention or distraction of the user, the user's mood, sociological dynamics associated with the user, user memories, and/or biophysical characteristics relevant in determining the cognitive state of the user.

In block 312, the mobile computing device 102 determines whether assistance is required based on the cognitive state of the user. For example, the mobile computing device 102 may identify a biophysical or physiological trait of the user that is indicative of cognitive impairment (e.g., that the user of the mobile computing device 102 is confused). If the mobile computing device 102 determines, in block 314, that the user does not require assistance, the method 300 returns to block 302 in which the mobile computing device 102 may establish a trust relationship with one or more remote computing devices 106. However, if the mobile computing device 102 determines that assistance is required, the mobile computing device 102 determines the type of assistance required in block 316. It should be appreciated that, in some embodiments, the mobile computing device 102 will determine whether the user requires assistance and the type of assistance required concurrently. As indicated above, in order to determine the type of assistance required, the mobile computing device 102 may, for example, compare data associated with the user's cognitive stated (e.g., the sensed data, biophysical characteristics, derived data, etc.) with various thresholds corresponding with the type of assistance required. In the illustrative embodiment, the types of assistance include assistance that can be provided without the involvement of a third party (i.e., may be provided by the mobile computing device 102), assistance that requires the involvement of a third party, and emergency assistance. However, in other embodiments, the types or levels of assistance may be otherwise partitioned or structured.

In block 318, the mobile computing device 102 determines whether the user requires assistance from another person. If not, the mobile computing device 102 provides cognitive assistance to the user in block 320. As discussed above, in doing so, the mobile computing device 102 may display a message to the user on the display(s) 134 in block 322 or generate an audible message to the user through the speaker(s) 132 in block 314. It should be appreciated that the particular message conveyed to the user may be any message suitable for remedying a cognitive issue the user is having. For example, if the user is having difficulty remembering a person's name, the message may include the name of the person and/or other information regarding the person. If the user is in a vehicle, the mobile computing device 102 may provide the user with directions (e.g., in real time) to the user's next location based on, for example, the user's stored schedule. Further, the mobile computing device 102 may default to returning the user to home if there is no indication that the user should be somewhere else.

If the mobile computing device 102 determines in block 318 that assistance is required from another person, the method 300 advances to block 326 of FIG. 4 in which the mobile computing device 102 identifies nearby computing devices 106 and/or persons. For example, the mobile computing device 102 may broadcast messages (e.g., ping) to all devices in the vicinity of the mobile computing device 102 and await responses to identify remote computing devices 106 in the vicinity of the mobile computing device 102. As discussed above, a reference distance (e.g., a communication range) may be utilized by the mobile computing device 102 to determine which computing devices 106 are “nearby” or “within the vicinity” of the mobile computing device 102. Additionally, the mobile computing device 102 may, for example, capture images (e.g., video) of the surrounding environment of the mobile computing device 102 and analyze those images to identify persons, if any, in the vicinity of the mobile computing device 102 (e.g., using facial identification and/or recognition techniques). In block 318, the mobile computing device 102 identifies any trusted computing devices 106 near the mobile computing device 102. As discussed above, the mobile computing device 102 may utilize stored identification data of trusted computing devices 106 for doing so. In particular, the mobile computing device 102 may compare the stored identification data to identification data of remote computing devices 106 identified near the mobile computing device 102 to detect a match.

In block 330, the mobile computing device 102 determines whether emergency assistance is required. If so, the mobile computing device 102 alerts nearby persons of the emergency in block 332. In doing so, the mobile computing device 102 may communicate with the nearby remote computing devices 106 in block 334 (e.g., transmit a message indicative of the emergency and identify the user of the mobile computing device 102) and/or generate an alert on the mobile computing device 102 in block 336. For example, the mobile computing device 102 may generate and audible alert through the speakers 132 or otherwise provide some audiovisual output to persons in the vicinity of the mobile computing device 102 that the user requires emergency assistance. As such, it should be appreciated that, in some embodiments, the mobile computing device 102 is indifferent as to which persons learn of the user's cognitive impairment if an emergency situation arises and, as such, may communicate with computing devices with which a trusted relationship has not been established. The method 300 returns to block 302 of FIG. 3 in which the mobile computing device 102 may determine whether to establish a trust relationship with a remote computing device 106.

If the mobile computing device 102 determines in block 330 that emergency assistance is not required, the mobile computing device 102 determines whether trusted device(s) and/or person(s) are near the mobile computing device 102 in block 338 (e.g., based on the determination of block 328). If so, the mobile computing device 102 notifies one or more of the trusted device(s) 106 that the user requires assistance (e.g., a simple alert message) and/or of the user's cognitive state (e.g., by transmitting data indicative of the user's cognitive state). In doing so, the mobile computing device 102 may establish a secure communication channel with the trusted device(s) 106 and securely communicate with the trusted device(s) 106 in block 344.

Returning to block 338, if the mobile computing device 102 determines there are no trusted computing devices 106 nearby, the mobile computing device 102 may notify one or more of the untrusted remote computing devices 106 near the mobile computing device 102 that the user requires assistance and/or the user's cognitive state in block 340 in some embodiments. In some embodiments, the mobile computing device 102 may provide such notification in a manner similar to that discussed above with respect to alerting nearby devices 106 and/or persons of an emergency. It should be appreciated that, in some embodiments, the mobile computing device 102 may transmit more information regarding the user and/or the user's cognitive state to trusted devices 106 than untrusted devices 106. For example, the mobile computing device 102 may transmit the sensor data, biophysical characteristics, and/or other information regarding the user's cognitive state to the trusted devices 106 but only an indication that the user needs assistance to the untrusted computing devices 106. It should further be appreciated that, in some embodiments, the mobile computing device 102 communicates with a subset and not all of the identified nearby computing devices 106. As such, the mobile computing device 102 may identify those in which to notify regarding the user's need of assistance according to any suitable scheme. For example, the mobile computing device 102 may select one or more computing devices 106 or trusted computing devices 106 nearest the mobile computing device 102 with which to communicate.

In block 346, the mobile computing device 102 determines whether instructions have been received from nearby devices 106. In other words, in some circumstances, the remote computing devices 106 may transmit instructions for how the mobile computing device 102 should address the user's need for assistance rather than the remote user communicating directly with the user of the mobile computing device 102. If not, the method 300 returns to block 302 of FIG. 3 in which the mobile computing device 102 may establish a trust relationship with a remote computing device 106. If instructions have been received, the mobile computing device 102 provides cognitive assistance to the user based on the received instructions in block 348. In particular, similar to that discussed above, the mobile computing device 102 may display a message to the user in block 350 and/or generate an audible message to the user in block 352 based on the received instructions. The method 300 returns to block 302 of FIG. 3 in which the mobile computing device 102 may establish a trust relationship with a remote computing device 106.

### EXAMPLES

Illustrative examples of the technologies disclosed herein are provided below. An embodiment of the technologies may include any one or more, and any combination of, the examples described below.

Example 1 includes a system for managing cognitive assistance provided to a user of the system, the system comprising one or more biosensors to generate sensor data indicative of a biophysical characteristic of the user; a cognitive state determination module to determine a cognitive state of the user based on the sensor data generated by the one or more biosensors; and a cognitive assistance module to (i) determine whether the user requires assistance based on the determined cognitive state of the user, (ii) identify, in response to a determination that the user requires assistance, a trusted mobile computing device within a vicinity of the system based on a trust relationship previously established between the system and the trusted mobile computing device, and (iii) communicate with the trusted mobile computing device to notify a remote user of the trusted mobile computing device that the user requires assistance.

Example 2 includes the subject matter of Example 1, and further including a wearable computing device, wherein the wearable computing device includes the one or more biosensors, the cognitive state determination module, and the cognitive assistance module.

Example 3 includes the subject matter of any of Examples 1 and 2, and further including a wearable computing device and a mobile computing device; wherein the wearable computing device includes the one or more biosensors; and wherein the mobile computing device includes the cognitive state determination module and the cognitive assistance module.

Example 4 includes the subject matter of any of Examples 1-3, and further including a mobile computing device, wherein the mobile computing device includes the one or more biosensors, the cognitive state determination module, and the cognitive assistance module.

Example 5 includes the subject matter of any of Examples 1-4, and wherein the cognitive assistance module is further to identify a plurality of remote computing devices within the vicinity of the system; and determine whether each remote computing device of the plurality of remote computing devices has established a trust relationship with the system; wherein to identify the trusted mobile computing device comprises to select a remote computing device from the plurality of remote computing devices that has established a trust relationship with the system.

Example 6 includes the subject matter of any of Examples 1-5, and wherein to select the remote computing device comprises to select the remote computing device of the plurality of remote computing devices nearest the system.

Example 7 includes the subject matter of any of Examples 1-6, and wherein to communicate with the trusted mobile computing device to notify the remote user comprises to communicate with each remote computing device that has established a trust relationship with the system to notify the corresponding remote user that the user requires assistance.

Example 8 includes the subject matter of any of Examples 1-7, and wherein to communicate with the trusted mobile computing device to notify the remote user that the user requires assistance comprises to notify the remote user of the user's cognitive state.

Example 9 includes the subject matter of any of Examples 1-8, and wherein to notify the remote user of the user's cognitive state comprises to transmit the sensor data to the trusted mobile computing device.

Example 10 includes the subject matter of any of Examples 1-9, and further including a trust establishment module to establish a trust relationship with the trusted mobile computing device.

Example 11 includes the subject matter of any of Examples 1-10, and wherein to establish the trust relationship comprises to store identification data of the trusted mobile computing device; and wherein to identify the trusted mobile computing device comprises to identify the trusted mobile computing device based on the received identification data.

Example 12 includes the subject matter of any of Examples 1-11, and wherein to determine the cognitive state of the user comprises to sense one or more biophysical characteristics of the user with the one or more biosensors; and analyze the one or more sensed biophysical characteristics to determine the user's cognitive state.

Example 13 includes the subject matter of any of Examples 1-12, and wherein to sense the one or more biophysical characteristics comprises to sense electromagnetic physical activity of the user.

Example 14 includes the subject matter of any of Examples 1-13, and wherein to sense the one or more biophysical characteristics comprises to optically sense the one or more biophysical characteristics of the user.

Example 15 includes the subject matter of any of Examples 1-14, and wherein to determine the user requires assistance comprises to determine a type of assistance required by the user based on the determined cognitive state of the user.

Example 16 includes the subject matter of any of Examples 1-15, and wherein to determine the type of assistance required comprises to compare the sensed biophysical characteristics to one or more thresholds.

Example 17 includes the subject matter of any of Examples 1-16, and wherein the cognitive assistance module is further to provide cognitive assistance to the user based on the determined cognitive state of the user and in response to a determination that the user does not require assistance from another person.

Example 18 includes the subject matter of any of Examples 1-17, and wherein to provide cognitive assistance to the user comprises to display a message on a display of the system directed to assisting the user.

Example 19 includes the subject matter of any of Examples 1-18, and wherein to provide cognitive assistance to the user comprises to render a message on a speaker of the system directed to assisting the user.

Example 20 includes the subject matter of any of Examples 1-19, and wherein to communicate with the trusted mobile computing device comprises to receive instructions from the trusted mobile computing device regarding providing cognitive assistance to the user; and wherein the cognitive assistance module is further to provide cognitive assistance to the user based on the received instructions.

Example 21 includes the subject matter of any of Examples 1-20, and wherein to provide the cognitive assistance comprises to display a message on a display of the system directed to assisting the user based on the received instructions.

Example 22 includes the subject matter of any of Examples 1-21, and wherein to provide the cognitive assistance comprises to render a message on a speaker of the system directed to assisting the user based on the received instructions.

Example 23 includes the subject matter of any of Examples 1-22, and wherein the cognitive assistance module is further to identify, in response to a determination that the user requires assistance from another person, a person within a reference range of the system.

Example 24 includes the subject matter of any of Examples 1-23, and wherein the cognitive assistance module is further to alert the person within the reference range of the system of an emergency related to the user in response to a determination that the user requires emergency assistance from another person.

Example 25 includes the subject matter of any of Examples 1-24, and wherein to identify the person within the reference range of the system comprises to identify a mobile computing device of the person within the reference range of the system.

Example 26 includes a method of managing cognitive assistance provided to a user of a cognitive assistance system, the method comprising determining, by the cognitive assistance system, a cognitive state of the user based on sensor data generated by one or more biosensors of the cognitive assistance system; determining, by the cognitive assistance system, whether the user requires assistance based on the determined cognitive state of the user; identifying, by the cognitive assistance system and in response to determining the user requires assistance, a trusted mobile computing device within a vicinity of the cognitive assistance system based on a trust relationship previously established between the cognitive assistance system and the trusted mobile computing device; and communicating, by the cognitive assistance system, with the trusted mobile computing device to notify a remote user of the trusted mobile computing device that the user requires assistance.

Example 27 includes the subject matter of Example 26, and wherein determining the cognitive state of the user comprises determining a cognitive state of the user based on sensor data generated by one or more biosensors of a wearable computing device.

Example 28 includes the subject matter of any of Examples 26 and 27, and wherein determining the cognitive state of the user comprises determining the cognitive state of the user by a mobile computing device different from the wearable computing device; determining whether the user requires assistance comprises determining, by the mobile computing device, whether the user requires assistance; identifying the trusted mobile computing device comprises determining, by the mobile computing device, the trusted mobile computing device within the vicinity of the cognitive assistance system; and communicating with the trusted mobile computing device comprises communicating, by the mobile computing device, with the trusted mobile computing device to notify the remote user of the trusted mobile computing device that the user requires assistance.

Example 29 includes the subject matter of any of Examples 26-28, and wherein determining the cognitive state of the user comprises determining the cognitive state of the user by the wearable computing device; determining whether the user requires assistance comprises determining, by the wearable computing device, whether the user requires assistance; identifying the trusted mobile computing device comprises determining, by the wearable computing device, the trusted mobile computing device within the vicinity of the cognitive assistance system; and communicating with the trusted mobile computing device comprises communicating, by the wearable computing device, with the trusted mobile computing device to notify the remote user of the trusted mobile computing device that the user requires assistance.

Example 30 includes the subject matter of any of Examples 26-29, and further including identifying, by the cognitive assistance system, a plurality of remote computing devices within the vicinity of the cognitive assistance system; and determining, by the cognitive assistance system, whether each remote computing device of the plurality of remote computing devices has established a trust relationship with the cognitive assistance system; wherein identifying the trusted mobile computing device comprises selecting a remote computing device from the plurality of remote computing devices that has established a trust relationship with the cognitive assistance system.

Example 31 includes the subject matter of any of Examples 26-30, and wherein selecting the remote computing device comprises selecting the remote computing device of the plurality of remote computing devices nearest the cognitive assistance system.

Example 32 includes the subject matter of any of Examples 26-31, and wherein communicating with the trusted mobile computing device to notify the remote user comprises communicating with each remote computing device that has established a trust relationship with the cognitive assistance system to notify the corresponding remote user that the user requires assistance.

Example 33 includes the subject matter of any of Examples 26-32, and wherein communicating with the trusted mobile computing device to notify the remote user that the user requires assistance comprises notifying the remote user of the user's cognitive state.

Example 34 includes the subject matter of any of Examples 26-33, and wherein notifying the remote user of the user's cognitive state comprises transmitting the sensor data to the trusted mobile computing device.

Example 35 includes the subject matter of any of Examples 26-34, and further including establishing, by the cognitive assistance system, a trust relationship with the trusted mobile computing device.

Example 36 includes the subject matter of any of Examples 26-35, and wherein establishing the trust relationship comprises storing identification data of the trusted mobile computing device; and identifying the trusted mobile computing device comprises identifying the trusted mobile computing device based on the received identification data.

Example 37 includes the subject matter of any of Examples 26-36, and wherein determining the cognitive state of the user comprises sensing one or more biophysical characteristics of the user with the one or more biosensors; and analyzing the one or more sensed biophysical characteristics to determine the user's cognitive state.

Example 38 includes the subject matter of any of Examples 26-37, and wherein sensing the one or more biophysical characteristics comprises sensing electromagnetic physical activity of the user.

Example 39 includes the subject matter of any of Examples 26-38, and wherein sensing the one or more biophysical characteristics comprises optically sensing the one or more biophysical characteristics of the user.

Example 40 includes the subject matter of any of Examples 26-39, and wherein determining the user requires assistance comprises determining a type of assistance required by the user based on the determined cognitive state of the user.

Example 41 includes the subject matter of any of Examples 26-40, and wherein determining the type of assistance required comprises comparing the sensed biophysical characteristics to one or more thresholds.

Example 42 includes the subject matter of any of Examples 26-41, and further including providing, by the cognitive assistance system, cognitive assistance to the user based on the determined cognitive state of the user and in response to determining the user does not require assistance from another person.

Example 43 includes the subject matter of any of Examples 26-42, and wherein providing cognitive assistance to the user comprises displaying a message on a display of the cognitive assistance system directed to assisting the user.

Example 44 includes the subject matter of any of Examples 26-43, and wherein providing cognitive assistance to the user comprises rendering a message on a speaker of the cognitive assistance system directed to assisting the user.

Example 45 includes the subject matter of any of Examples 26-44, and wherein communicating with the trusted mobile computing device comprises receiving instructions from the trusted mobile computing device regarding providing cognitive assistance to the user; and further comprising providing, by the cognitive assistance system, cognitive assistance to the user based on the received instructions.

Example 46 includes the subject matter of any of Examples 26-45, and wherein providing the cognitive assistance comprises displaying a message on a display of the cognitive assistance system directed to assisting the user based on the received instructions.

Example 47 includes the subject matter of any of Examples 26-46, and wherein providing the cognitive assistance comprises rendering a message on a speaker of the cognitive assistance system directed to assisting the user based on the received instructions.

Example 48 includes the subject matter of any of Examples 26-47, and further including identifying, by the cognitive assistance system and in response to determining the user requires assistance from another person, a person within a reference range of the cognitive assistance system.

Example 49 includes the subject matter of any of Examples 26-48, and further including alerting, by the cognitive assistance system, the person within the reference range of the cognitive assistance system of an emergency related to the user in response to determining the user requires emergency assistance from another person.

Example 50 includes the subject matter of any of Examples 26-49, and wherein identifying the person within the reference range of the cognitive assistance system comprises identifying a mobile computing device of the person within the reference range of the cognitive assistance system.

Example 51 includes a computing device comprising a processor; and a memory having stored therein a plurality of instructions that when executed by the processor cause the computing device to perform the method of any of Examples 26-50.

Example 52 includes one or more machine-readable storage media comprising a plurality of instructions stored thereon that, in response to execution by a cognitive assistance system, cause the cognitive assistance system to perform the method of any of Examples 26-50.

Example 53 includes a cognitive assistance system for managing cognitive assistance provided to a user of the cognitive assistance system, the cognitive assistance system comprising means for determining a cognitive state of the user based on sensor data generated by one or more biosensors of the cognitive assistance system; means for determining whether the user requires assistance based on the determined cognitive state of the user; means for identifying, in response to determining the user requires assistance, a trusted mobile computing device within a vicinity of the cognitive assistance system based on a trust relationship previously established between the cognitive assistance system and the trusted mobile computing device; and means for communicating with the trusted mobile computing device to notify a remote user of the trusted mobile computing device that the user requires assistance.

Example 54 includes the subject matter of Example 53, and wherein the means for determining the cognitive state of the user comprises means for determining a cognitive state of the user based on sensor data generated by one or more biosensors of a wearable computing device.

Example 55 includes the subject matter of any of Examples 53 and 54, and wherein the means for determining the cognitive state of the user comprises means for determining the cognitive state of the user by a mobile computing device different from the wearable computing device; the means for determining whether the user requires assistance comprises means for determining, by the mobile computing device, whether the user requires assistance; the means for identifying the trusted mobile computing device comprises means for determining, by the mobile computing device, the trusted mobile computing device within the vicinity of the cognitive assistance system; and the means for communicating with the trusted mobile computing device comprises means for communicating, by the mobile computing device, with the trusted mobile computing device to notify the remote user of the trusted mobile computing device that the user requires assistance.

Example 56 includes the subject matter of any of Examples 53-55, and wherein the means for determining the cognitive state of the user comprises means for determining the cognitive state of the user by the wearable computing device; the means for determining whether the user requires assistance comprises means for determining, by the wearable computing device, whether the user requires assistance; the means for identifying the trusted mobile computing device comprises means for determining, by the wearable computing device, the trusted mobile computing device within the vicinity of the cognitive assistance system; and the means for communicating with the trusted mobile computing device comprises means for communicating, by the wearable computing device, with the trusted mobile computing device to notify the remote user of the trusted mobile computing device that the user requires assistance.

Example 57 includes the subject matter of any of Examples 53-56, and further including means for identifying a plurality of remote computing devices within the vicinity of the cognitive assistance system; and means for determining whether each remote computing device of the plurality of remote computing devices has established a trust relationship with the cognitive assistance system; wherein the means for identifying the trusted mobile computing device comprises means for selecting a remote computing device from the plurality of remote computing devices that has established a trust relationship with the cognitive assistance system.

Example 58 includes the subject matter of any of Examples 53-57, and wherein the means for selecting the remote computing device comprises means for selecting the remote computing device of the plurality of remote computing devices nearest the cognitive assistance system.

Example 59 includes the subject matter of any of Examples 53-58, and, wherein the means for communicating with the trusted mobile computing device to notify the remote user comprises means for communicating with each remote computing device that has established a trust relationship with the cognitive assistance system to notify the corresponding remote user that the user requires assistance.

Example 60 includes the subject matter of any of Examples 53-59, and wherein the means for communicating with the trusted mobile computing device to notify the remote user that the user requires assistance comprises means for notifying the remote user of the user's cognitive state.

Example 61 includes the subject matter of any of Examples 53-60, and wherein the means for notifying the remote user of the user's cognitive state comprises means for transmitting the sensor data to the trusted mobile computing device.

Example 62 includes the subject matter of any of Examples 53-61, and further including means for establishing a trust relationship with the trusted mobile computing device.

Example 63 includes the subject matter of any of Examples 53-62, and wherein the means for establishing the trust relationship comprises means for storing identification data of the trusted mobile computing device; and the means for identifying the trusted mobile computing device comprises means for identifying the trusted mobile computing device based on the received identification data.

Example 64 includes the subject matter of any of Examples 53-63, and wherein the means for determining the cognitive state of the user comprises means for sensing one or more biophysical characteristics of the user with the one or more biosensors; and means for analyzing the one or more sensed biophysical characteristics to determine the user's cognitive state.

Example 65 includes the subject matter of any of Examples 53-64, and wherein the means for sensing the one or more biophysical characteristics comprises means for sensing electromagnetic physical activity of the user.

Example 66 includes the subject matter of any of Examples 53-65, and wherein the means for sensing the one or more biophysical characteristics comprises means for optically sensing the one or more biophysical characteristics of the user.

Example 67 includes the subject matter of any of Examples 53-66, and wherein the means for determining the user requires assistance comprises means for determining a type of assistance required by the user based on the determined cognitive state of the user.

Example 68 includes the subject matter of any of Examples 53-67, and wherein the means for determining the type of assistance required comprises means for comparing the sensed biophysical characteristics to one or more thresholds.

Example 69 includes the subject matter of any of Examples 53-68, and further including means for providing cognitive assistance to the user based on the determined cognitive state of the user and in response to determining the user does not require assistance from another person.

Example 70 includes the subject matter of any of Examples 53-69, and wherein the means for providing cognitive assistance to the user comprises means for displaying a message on a display of the cognitive assistance system directed to assisting the user.

Example 71 includes the subject matter of any of Examples 53-70, and wherein the means for providing cognitive assistance to the user comprises means for rendering a message on a speaker of the cognitive assistance system directed to assisting the user.

Example 72 includes the subject matter of any of Examples 53-71, and wherein the means for communicating with the trusted mobile computing device comprises means for receiving instructions from the trusted mobile computing device regarding providing cognitive assistance to the user; and further comprising means for providing cognitive assistance to the user based on the received instructions.

Example 73 includes the subject matter of any of Examples 53-72, and wherein the means for providing the cognitive assistance comprises means for displaying a message on a display of the cognitive assistance system directed to assisting the user based on the received instructions.

Example 74 includes the subject matter of any of Examples 53-73, and wherein the means for providing the cognitive assistance comprises means for rendering a message on a speaker of the cognitive assistance system directed to assisting the user based on the received instructions.

Example 75 includes the subject matter of any of Examples 53-74, and further including means for identifying, in response to determining the user requires assistance from another person, a person within a reference range of the cognitive assistance system.

Example 76 includes the subject matter of any of Examples 53-75, and further including means for alerting the person within the reference range of the cognitive assistance system of an emergency related to the user in response to determining the user requires emergency assistance from another person.

Example 77 includes the subject matter of any of Examples 53-76, and, wherein the means for identifying the person within the reference range of the cognitive assistance system comprises means for identifying a mobile computing device of the person within the reference range of the cognitive assistance system.

1. A system for managing cognitive assistance provided to a user of the system, the system comprising:
one or more biosensors to generate sensor data indicative of a biophysical characteristic of the user;
a cognitive state determination module to determine a cognitive state of the user based on the sensor data generated by the one or more biosensors; and
a cognitive assistance module to (i) determine whether the user requires assistance based on the determined cognitive state of the user, (ii) identify, in response to a determination that the user requires assistance, a trusted mobile computing device within a vicinity of the system based on a trust relationship previously established between the system and the trusted mobile computing device, and (iii) communicate with the trusted mobile computing device to notify a remote user of the trusted mobile computing device that the user requires assistance.

2. The system of claim 1, further comprising a wearable computing device, wherein the wearable computing device includes the one or more biosensors, the cognitive state determination module, and the cognitive assistance module.

3. The system of claim 1, further comprising a wearable computing device and a mobile computing device;
wherein the wearable computing device includes the one or more biosensors; and
wherein the mobile computing device includes the cognitive state determination module and the cognitive assistance module.

4. The system of claim 1, further comprising a mobile computing device, wherein the mobile computing device includes the one or more biosensors, the cognitive state determination module, and the cognitive assistance module.

5. The system of claim 1, wherein the cognitive assistance module is further to:
identify a plurality of remote computing devices within the vicinity of the system; and
determine whether each remote computing device of the plurality of remote computing devices has established a trust relationship with the system;
wherein to identify the trusted mobile computing device comprises to select a remote computing device from the plurality of remote computing devices that has established a trust relationship with the system.

6. The system of claim 5, wherein to communicate with the trusted mobile computing device to notify the remote user comprises to communicate with each remote computing device that has established a trust relationship with the system to notify the corresponding remote user that the user requires assistance.

7. The system of claim 1, wherein to communicate with the trusted mobile computing device to notify the remote user that the user requires assistance comprises to notify the remote user of the user's cognitive state.

8. The system of claim 7, wherein to notify the remote user of the user's cognitive state comprises to transmit the sensor data to the trusted mobile computing device.

9. The system of claim 1, wherein to determine the cognitive state of the user comprises to:
sense one or more biophysical characteristics of the user with the one or more biosensors; and
analyze the one or more sensed biophysical characteristics to determine the user's cognitive state.

10. The system of claim 9, wherein to sense the one or more biophysical characteristics comprises to sense electromagnetic physical activity of the user.

11. The system of claim 9, wherein to sense the one or more biophysical characteristics comprises to optically sense the one or more biophysical characteristics of the user.

12. The system of claim 1, wherein to determine the user requires assistance comprises to determine a type of assistance required by the user based on the determined cognitive state of the user.

13. The system of claim 12, wherein the cognitive assistance module is further to provide cognitive assistance to the user based on the determined cognitive state of the user and in response to a determination that the user does not require assistance from another person.

14. The system of claim 13, wherein to provide cognitive assistance to the user comprises to display a message on a display of the system or to render a message on a speaker of the system directed to assisting the user.

15. The system of claim 1, wherein the cognitive assistance module is further to identify, in response to a determination that the user requires assistance from another person, a person within a reference range of the system.

16. The system of claim 15, wherein the cognitive assistance module is further to alert the person within the reference range of the system of an emergency related to the user in response to a determination that the user requires emergency assistance from another person.

17. The system of claim 15, wherein to identify the person within the reference range of the system comprises to identify a mobile computing device of the person within the reference range of the system.

18. One or more machine-readable storage media comprising a plurality of instructions stored thereon that, in response to execution by a cognitive assistance system, cause the cognitive assistance system to:
determine a cognitive state of the user based on sensor data generated by one or more biosensors of the cognitive assistance system;
determine whether the user requires assistance based on the determined cognitive state of the user;
identify, in response to a determination that the user requires assistance, a trusted mobile computing device within a vicinity of the cognitive assistance system based on a trust relationship previously established between the cognitive assistance system and the trusted mobile computing device; and
communicate with the trusted mobile computing device to notify a remote user of the trusted mobile computing device that the user requires assistance.

19. The one or more machine-readable storage media of claim 18, wherein to determine the cognitive state of the user comprises to determine a cognitive state of the user based on sensor data generated by one or more biosensors of a wearable computing device.

20. The one or more machine-readable storage media of claim 18, wherein the plurality of instructions further cause the cognitive assistance system to:
identify a plurality of remote computing devices within the vicinity of the cognitive assistance system; and
determine whether each remote computing device of the plurality of remote computing devices has established a trust relationship with the cognitive assistance system;
wherein to identify the trusted mobile computing device comprises to select a remote computing device from the plurality of remote computing devices that has established a trust relationship with the cognitive assistance system.

21. The one or more machine-readable storage media of claim 18, wherein to determine the cognitive state of the user comprises to:
sense one or more biophysical characteristics of the user with the one or more biosensors; and
analyze the one or more sensed biophysical characteristics to determine the user's cognitive state.

22. The one or more machine-readable storage media of claim 18, wherein to determine the user requires assistance comprises to determine a type of assistance required by the user based on the determined cognitive state of the user.

23. A method of managing cognitive assistance provided to a user of a cognitive assistance system, the method comprising:
determining, by the cognitive assistance system, a cognitive state of the user based on sensor data generated by one or more biosensors of the cognitive assistance system;
determining, by the cognitive assistance system, whether the user requires assistance based on the determined cognitive state of the user;
identifying, by the cognitive assistance system and in response to determining the user requires assistance, a trusted mobile computing device within a vicinity of the cognitive assistance system based on a trust relationship previously established between the cognitive assistance system and the trusted mobile computing device; and
communicating, by the cognitive assistance system, with the trusted mobile computing device to notify a remote user of the trusted mobile computing device that the user requires assistance.

24. The method of claim 23, wherein determining the cognitive state of the user comprises determining a cognitive state of the user based on sensor data generated by one or more biosensors of a wearable computing device.

25. The method of claim 23, further comprising:
identifying, by the cognitive assistance system, a plurality of remote computing devices within the vicinity of the cognitive assistance system; and
determining, by the cognitive assistance system, whether each remote computing device of the plurality of remote computing devices has established a trust relationship with the cognitive assistance system;
wherein determining the cognitive state of the user comprises (i) sensing one or more biophysical characteristics of the user with the one or more biosensors and (ii) analyzing the one or more sensed biophysical characteristics to determine the user's cognitive state; and
wherein identifying the trusted mobile computing device comprises selecting a remote computing device from the plurality of remote computing devices that has established a trust relationship with the cognitive assistance system.
